(19) United States
(12) Patent Application Publication
(10) Pub. No.: US 2001/0044108 A1
(43) Pub. Date: Nov. 22, 2001

(54) Detection of biomolecules in gels following electrophoresis

(76) Inventors: Lih-Bin Shih, San Diego, CA (US); Mark Williams, Redwood Shores, CA (US); Patricia M. Vilalta, San Diego, CA (US); Yong-Min Yang, San Diego, CA (US)

Correspondence Address: Barry S. Wilson, Foley & Lardner, 23rd Floor, 402 West Broadway, San Diego, CA, 92101-3542, US

(21) Appl. No.: 09/771,304

(22) Filed: Jan. 26, 2001

Related U.S. Application Data
(60) Provisional application No. 60/178,820, filed on Jan. 28, 2000.

(51) Int. Cl. C12Q 1/68; G01N 33/53; G01N 33/537; G01N 33/543; G01N 33/557

(52) U.S. Cl. 435/6; 435/7.92; 436/517; 435/7.5

(57) ABSTRACT

The present invention comprises method of detecting biomolecules in situ in gel separation medium following electrophoresis that provides sensitivity similar to blotting. This method is applicable to in situ detection of a wide range of biomolecules including proteins and nucleic acids. After electrophoretic separation of a sample in gel separation media comprising a gellable polymeric material other than cross-linked polyacrylamide, the gel is contacted with a solution comprising at least one detectably labeled reagent directed to a biomolecule under conditions suitable for binding of the reagent to the biomolecule. Alternatively, the gel is contacted with a solution comprising at least one non-detectably labeled reagent directed to a biomolecule and a solution comprising at least on detectably labeled reagent directed to a biomolecule. In either case, binding of the detectably labeled reagent is assessed and indicates detection of biomolecules in the gel.

### FIELD OF THE INVENTION

The present invention relates generally to a method for detecting the presence of biomolecules in gels and, more specifically, to a method for detecting biomolecules directly in gels following electrophoresis.

### BACKGROUND OF THE INVENTION

Gel electrophoresis is a long standing well known method to analyze the complexity of a given preparation of biomolecules such as proteins or nucleic acids. When samples are subject to gel electrophoresis, biomolecules move at different rates in the gel depending on their charge and to some extent, their molecular size depending on the conditions. If gel conditions are properly chosen, the complexity and relative amount of different biomolecules in a sample can be revealed as individual bands of material the position of which in the gel relates to charge and/or size.

Electrophoresis of proteins in gels of cross-linked polyacrylamide or electrophoresis nucleic acids in agarose gels are is well known methods for analysis of these biomolecules. Biomolecules have long been detected in gels by direct staining such as with small molecular weight dyes. However, to detect a characteristic of a protein such as the presence of an antigenic determinant or the presence of a particular nucleotide sequence in a nucleic acid, gel electrophoresis has been commonly followed by blotting (i.e., transferring) the biomolecules from the gel to a membrane where detection is performed. Blotting is used for this purpose because the gel matrix presents significant barriers to reagent diffusion. For example, the thermoset polymer characteristic with permanent network structure of a cross-linked polyacrylamide gel limits passive diffusion of large biomolecules (e.g. antibodies or oligo probes) through the confined spaces in the gel (pore size as defined by a gel network). In contrast, blotting from the gel to a membrane eliminates any diffusion problem and improves sensitivity.

Blotting methods used in conjunction with gel electrophoresis are, however, associated with well known limitations. For example, the transfer rate of different biomolecules from gels to the blotting matrix varies according to the molecules' physical characteristics such as molecular weight, charge, and hydrophobicity. Determining appropriate transfer times and conditions must be accomplished empirically for each protein, often in the absence of data and without knowledge of the efficiency of detection. In addition, preparing the gel and blotting matrix for transfer and performing the transfer are time-consuming tasks—the gels and the blotting matrices must each be incubated in solutions to prepare them for transfer and a gel/matrix “sandwich” must be carefully assembled with filter papers in the transfer apparatus, either for passive (solution wicking) or active (electrophoresis in an orientation transverse to separation) transfer. Furthermore, blotting can take hours to overnight to complete, depending on the application and characteristics of the target molecules. Also, experiments may be lost during the extensive handling required for blotting because of the fragility of gels and membranes.

The inventors have surprisingly discovered that particular gel matrices and conditions of detection allow in situ gel detection of biomolecules using large molecular weight reagents such as an antibody molecule. This method avoids the problems associated with blotting methods and can be comparable in sensitivity.

### SUMMARY OF THE INVENTION

Accordingly, the present invention comprises method of detecting biomolecules in situ in gel separation medium following electrophoresis that provides sensitivity similar to blotting. This method is applicable to in situ detection of a wide range of biomolecules including proteins and nucleic acids.

The method comprises electrophoresing a sample of biomolecules in gel separation media comprising a gellable polymeric material other than cross-linked polyacrylamide. The gel separation media is then contacted with a solution comprising at least one detectably labeled reagent directed to a biomolecule under conditions suitable for binding of the reagent to the biomolecule or is contacted with a solution comprising at least one non-detectably labeled reagent directed to a biomolecule under conditions suitable for binding of the reagent to the biomolecule. Alternatively, the gel separation media is contacted with a solution comprising at least one detectably labeled reagent directed to the non-detectably labeled reagent under conditions suitable for binding of the detectably labeled reagent to the non-detectably labeled reagent. In either case, detection of binding of the detectably labeled reagent thus indicates detection of biomolecules in the gel.

The characteristics of various gellable materials useful for in situ detection are provided as well as various conditions that allow the method to be comparable in sensitivity to blotting of small molecular weight proteins.

### DETAILED DESCRIPTION OF THE INVENTION

In accordance with these and other embodiments of the present invention, there is provided a method for detecting biomolecules in situ following gel electrophoresis. The inventors have discovered surprisingly that in situ detection of biomolecules in gels using large molecular weight reagents provides a level of sensitivity comparable to blotting methods such as Western blotting where detection by a reagent is performed outside the gel on a membrane to which biomolecules in the gel have been transferred. In situ gel detection as performed herein can achieve detection of 10 nanograms or less of a protein.

The method of the invention can be used to detect an array of biomolecules, including, for example, proteins including polypeptides and peptides, nucleic acids including DNA, RNA, polynucleotides and oligonucleotides, carbohydrates, lipids, glycolipids, glycoproteins and proteoglycans, and charged polymine materials (both natural or synthetic). These terms have well known meanings in the art. Protein includes one or more chains of amino acids linked by peptide bonds. The term protein includes the term polypeptide, which refers to a single chain of amino acids and the term peptide which generally refers to a single chain of amino acids using less than about 50 amino acids in length. The term protein as used herein also encompasses glycoprotein, lipoprotein or proteoglycan type biomolecules, all of which include protein in their composition in addition to other material.

“Nucleic acid” or “polynucleotide” is a polymer of nucleotides, either single or double stranded. A polynucleotide will typically refer to a nucleic acid molecule comprising a linear strand of two or more deoxyribonucleotides and/or ribonucleotides. As used herein “polynucleotide” and its grammatical equivalents include the full range of nucleic acids including primers, probes, RNA/DNA segments, oligonucleotides or “oligos” (relatively short polynucleotides), genes, vectors, plasmids, and the like. An “oligonucleotide” refers to a relatively short polynucleotide.

“Carbohydrates” refer to sugar-based compounds containing carbon, hydrogen and oxygen with the general formula Cₓ (H₂O)_{y}. Carbohydrates can be divided into various sub-groups, i.e., monosaccharides, disaccharides, oligosaccharides or polysaccharides, depending on the degree of polymerization of the basic sugar units. As employed herein, “oligosaccharides” refer to carbohydrates containing a few monosaccharides.

“Lipids” refer to those compounds found in living organisms which are not carbohydrates, proteins or polynucleic acids. Lipids tend to be soluble in organic solvents and insoluble in water, and include fats, waxes, phospholipids, glycolipids, steroids, terpenes and a number of different types of pigments. The major group of lipids contains those compounds whose structure is characterized by the presence of fatty acid moieties (acyl lipids). These include neutral lipids (glycerides and waxes) and polar lipids (phospholipids and glycolipids). Glycolipids refer to lipids that contain one or more carbohydrate moieties. These lipids include the cerebrosides and gangliosides in animals and the galactosyl diglycerides and sulpholipids in plants. The lipid portion is usually glycerol phosphate, glycerol or sphingosine, and the carbohydrate is D-galactose, inositol or D-glucose.

The method of the invention is applicable to a wide variety of gels including those formed with a gellable polymeric material other than cross-linked polyacrylamide. A preferred gel is a hydrogel, prepared from thermoplastic polymers consisting of hydrophobic and hydrophilic blocks. Such a hydrogel is not covalently cross linked and it is subject to dissolution in particular solvents. As discovered herein, the gel network of such hydrogels can be manipulated with electrolyte solutions and/or the presence of co-solvents to achieve high sensitivity in situ detection using large molecular weight reagents. The choice of electrolyte solutions (the type, the ionic strength and the pH) and/co solvents controls the gel pore size and the interactions of the gel matrix with the biological molecules. As a result, the diffusion of large molecule reagents into the gel to detect biomolecules is no longer problematic.

The in situ gel detection method avoids the time and expense associated with blotting following gel electrophoresis that previously has been considered a necessary step to biomolecule detection using large molecular weight reagents such as antibodies. With in situ detection, one avoids the need to determine transfer efficiency for each species of biomolecule for blotting and avoids the costs of purchasing expensive electrophoretic transfer devices. The relatively high mechanical strength of the gellable materials used herein, particularly polyacetonitrile/polyacrylamide copolymer gel materials, provides for easy handling during incubations with reagent and archiving of data. In addition, the gellable material used herein can be chosen to have very low affinity for the agent used for in situ detection. This contrasts with detection in conventional blotting methods where the membrane or paper-like matrix used in blotting has natural affinity both for the transferred molecules and for the agent used in detection (e.g. nitrocellulose has affinity for both protein and the antibody used to detect the protein). Thus, the in situ detection method herein can be designed so as to avoid a blocking step.

The gellable polymeric material used in gels for in situ detection of biomolecules provides a number of advantages over other polymers employed in the art. For example, the gel pore size of gellable polymeric material can be readily adjusted by adjusting the degree of hydrophobic and hydrophillic balance, the extent of chain entanglement, the degree of cohesive dipolar forces in the polymeric material, and the electrolyte conditions. Such materials have good mechanical strength for repeated handling, a feature useful for in situ biomolecule detection. Gellable polymeric materials used for gels herein are stable in the presence of a wide range of conditions including a wide range of temperature, pH and the like without concern for degradation. These materials can reproducibly be manufactured to exacting specifications on large scale and can be precasted. Gellable polymeric materials as used herein are preferably synthetically prepared.

The gellable polymeric material used for in situ detection following electrophoresis is prepared in an aqueous medium prior to gel formation. Aqueous media include saline, buffered aqueous media having a pH in the range of about 2 up to 12, aqueous solutions of lower alcohols, aqueous surfactant-containing solutions, aqueous solutions containing salt or other electrolytes, and the like. For separation of high molecular weight biomolecules, the gellable material will generally contain in the range of about 50 up to 99.5 wt % aqueous medium. At such high water contents, the pore size of resulting gel will be maximized. Larger pores made possible by such high water content provides a sieving action for larger (i.e., high molecular weight) molecules. For smaller size biomolecules, the gellable material will generally contain in the range of about 20 up to 85 wt % aqueous medium. At such water levels, pore sizes in the separation gel will be proportionately reduced, thereby providing a sieving action for smaller molecules.

The structural integrity of gels used herein also can be imparted by chemical modification of the gellable material by, for example, chemical linking of polymer chains (e.g., covalent cross-linking, or ionic bonding cross-linking), physical interaction of (e.g., hydrogen bonding of polymer chains, hydrophobic interactions (such as the presence of crystalline domains), physical entanglement of polymer chains, and hydrophobic interactions including dipolar forces, etc. and the like. Where dipolar forces make a significant contribution to the structural integrity of the gellable polymeric material, the pore size of the gel can be varied by appropriate modification of the chemical structure of the polymer, as well as manipulation of the electrolyte conditions (i.e., ionic strength, buffer type and pH and addition of co-solvents).

Cross-linking agents include bifunctional compounds which serve to bridge two different polymer chains. Commonly used cross-linking agents are alpha- or omega-diolefins, which are incorporated into the forming polymer by free radical polymerization. The degree of cross-linking imparted to the gellable material impacts the pore size achievable by the resulting resin. When chemical cross-linking agents are not used for the preparation of gellable polymeric material, gel pore size can be controlled by controlling the extent the gellable polymeric material is capable of chain entanglement and cohesive or other hydrophobic interactions including dipolar forces, and by controlling the electrolyte conditions (e.g., ionic strength, pH and buffer type) employed for the separation process. Thus, the longer the chain length of the polymer backbone between chemical cross-links and/or chain entanglement points, the longer the potential pore size obtainable by the resulting gel. Where the gellable polymeric material employed in the practice of the present invention forms a hydrogel, based at least in part upon cohesive dipolar forces, the gel pore size can be varied by appropriate manipulation of the electrolyte conditions (e.g., ionic strength, buffer type and pH). Gellable polymeric materials useful in methods of the present invention may be prepared with our without covalent cross-linking. In addition, gradient gels also my be used in the methods of the present invention.

Gellable material is generally prepared in a support having deposited thereon a layer of about 0.15-5 mm thickness of the gellable material. Support materials include glass plates, plastic sheets, and the like. Alternatively, gellable material can be incorporated into support structures such as columns, glass tubing, capillary tubing, glass cells, and the like. Suitable support structures can be constructed of a variety of materials, as can be readily determined by those of skill in the art (e.g., glass, plastic, and the like). It is understood that gels will need to be removed from support structures to the extent necessary to provide access to reagents for in situ detection as described herein.

Exemplary gellable polymeric materials useful herein include chemically cross-linked polymers other than cross-linked polyacrylamide such as N-vinyl pyrrolidone-based polymers, methacrylic acid-based polymers (e.g., glyceryl methacrylate-based polymers, 2-hydroxyethylmethylacrylate-based polymers, and the like), acrylic acid-based polymers, and the like, containing hydrophilic groups such as hydroxy, amine, and the like; physically entangled polymers and polymer networks formed by cohesive dipolar forces, such as, for example, multi-block copolymers as described in U.S. Pat. No. 5,888,365 to Shih et al. These materials share various properties including: (i) ability to form gels having an aqueous content range from about 20 up to 99.5 wt %; (ii) having hydrophilic characteristics with a controllable degree of hydrophilicity; and (iii) having sufficient strength, in the presence of high levels of aqueous media, to retain its structural integrity.

The preparation of gels suitable for use in the present invention is described in detail in U.S. Pat. No. 5,388,365 to Shih and in the Examples below. Following electrophoresis, the gels may optionally be “fixed” to reduce diffusion of gel bands during the various gel processing steps (incubation and washings) used for in situ detection. Fixation conditions should be chosen to avoid interfering with subsequent binding by antibody or other binding agent (e.g. nucleic acid). Fixation can be accomplished with any of a variety of solvents and co-solvents. A gel fixation solution can include an aqueous solution comprising an alcohol, an acid or an organic solvent or co-solvent, or combinations of the above. Suitable fixing solutions include, for example, aqueous mixtures of ethanol, an organic solvent, an acid such as trichloroacetic acid (TCA) or acetic acid or combinations thereof. Exemplary gel fixation solutions include 10% TCA/40% methanol, or 20%-50% ethanol with 5-10% acetic acid, the latter being preferred.

Before gels are contacted with a reagent (e.g., an antibody), the gel optionally may be treated with a blocking solution to reduce background binding of the reagent to the gel. Blocking solutions are well known in the art and include, for example, solutions containing albumin, serum, nonfat dry milk (i.e., “blotto”) and nonionic detergents such as Tween 20, and various combinations of the above. See e.g., Harlow and Lane, Antibodies: A Laboratory Manual, Cold Spring Harbor Laboratory Press, 1988. A solution containing 3% non-fat dry milk/0.1×TBN (300 mM Tris Base; and 129 mM boric acid) is preferred for reducing background binding in in situ detection. Substances used in blocking solutions also can be included in the diluent used for reagents to further eliminate background binding. A preferred reagent diluent with blocking is 0.1×TBN (300 mM Tris Base; and 129 mM Boric Acid)/1.5% non-fat dry milk.

The time required to achieve fixation can vary from a few minutes to days, depending in part on the choice of fixative. Using the preferred fixatives above, in situ immunological detection can be observed using between 10 to 30 minutes fixation time for protein ranging from 200,000 Daltons (kDa) to 6.5 kDa.

A used herein a “reagent” is any substance that has binding specificity for a biomolecule. “Specific binding” means that the reagent detectably binds to some biomolecules, but not to all biomolecules. A reagent includes, for example, an antibody, avidin, streptavidin, oligonucleotide probe and the like. An “antibody” can be any of a large number of proteins of high molecular weight that are produced normally by specialized B type lymphocytes after stimulation by an antigen and act specifically against the antigen in an immune response. The term antibody also encompasses naturally occurring antibodies as well as non-naturally occurring antibodies such as domain-deleted antibodies, single chain Fv antibodies and the like. Reagents useful for in situ detection range in size from as low as about 5 kDa (e.g. a small oligonucleotide) to greater than 150 kDa (e.g. an antibody).

As used herein a “second reagent” is a substance that has binding specificity for a first reagent. A second reagent can be an antibody that is specific for the first antibody. An example of a second antibody is a goat anti-mouse antibody where in the first antibody is a mouse antibody. Avidin or streptavidin, which have binding specificity for biotin can be considered second reagents as used herein if the first reagent is labeled with biotin. In addition, an antibody can be a second antibody if it is specific for a hapten which has been conjugated to a first reagent. Anti-hapten antibodies such as those directed to pbosphorylcholine or dinitrophenol are well known in the art and are commercially available.

The first or the second reagent can be labeled with a detectable moiety to provide the ability to visualize binding of the reagent to a biomolecule in the gel. As used herein, the term “detectably labeled” used in reference to a reagent includes, for example, reagents labeled with a detectable moiety such as a radioisotope, an enzyme, a fluorochrome or dye, a hapten, or a small chemical such as biotin. A variety of detectable moieties and methods to conjugate such moieties to a reagent are well known in the art. See e.g., Harlow and Lane, supra, 1988. Detectable moieties include radioisotopes such as ¹²⁵I and ¹³¹I, enzymes such as horseradish peroxidase, alkaline phosphatase and β-galactosidase, fluorochromes or dyes such as fluorescein, rhodamine and the like. Various substrates useful to visualize the presence of reagent-enzyme conjugate bound in situ to biomolecules in a gel also are well known in the art. Harlow and Lane, supra, 1988.

In situ detection also can be applied to the detection of nucleic acids in gels by hybridization with poly- or oligonucleotide probe reagents. As used herein, “hybridization:” is the pairing of substantially complementary nucleotide sequences (strands of nucleic acid) to form a duplex or heteroduplex through formation of hydrogen bonds between complementary base pairs. It is a specific, i.e., non-random, interaction between two complementary polynucleotides. Hybridization stringency refers to the conditions under which hybridization between two nucleic acid strands is conducted. High stringency refers to conditions that permit hybridization of only those nucleic acid sequences that form stable hybrids in 0.018 M NaCl at 65° C. High stringency conditions can be provided, for example, by hybridization in 50% formamide, 5× Denhardt's solution, 5× sodium chloride- sodium phosphate-Ethylenediaminetretracetic acid buffer (SSPE buffer), 0.2% sodium dodecyl sulfate (SDS) at 42° C., followed by washing in 0.1× SSPE, and 0.1% SDS at 65° C. Moderate stringency refers to conditions equivalent to hybridization in 50% formamide, 5× Denhardt's solution, 5× SSPE, 0.2% SDS at 42° C., followed by washing in 0.2× SSPE, 0.2% SDS, at 65° C. Low stringency refers to conditions equivalent to hybridization in 10% formamide, 5× Denhardt's solution, 6× SSPE, 0.2% SDS, followed by washing in 1× SSPE, 0.2% SDS, at 50° C. Recipes for Denhardt's solution and SSPE are well known to those of skill in the art as are other suitable hybridization buffers (e.g., Sambrook et al., Molecular Cloning: A Laboratory Manual, CSH Laboratory Press, Cold Spring Harbor, N.Y. 1989).

### EXAMPLES

### Example 1

Procedures for In Situ gel Detection of Biomolecules

This example describes experimental details to perform various steps in the method of the invention. The details of each step are merely exemplary. One skilled in the art will be able to modify concentrations, temperature of incubation and time of incubation as desired for each situation. Also, one skilled in the art would be familiar with a variety of buffers and solvents other than those mentioned below that can be successfully used in the method.

(a) Gel Preparation and Electrophoresis:

Gels referred to herein as “AHT hydrogels” are prepared in two steps. The first step is hydrolysis of linear polyacylonitrile polymer (Scientific Polymer Products, Inc. Ontario N.Y., CAS#. 25014-41-9, Cat no. 134) under acidic conditions as described, for example, in U.S. Pat. No. 3,948,870 to Stoy et al.) Following hydrolysis, the gel is neutralized, dried and dissolved in polar solvents such as DMF or DMSO at a certain weight concentration. In the second step, the polymer solution is cast into a slab gel as described in Shih et al., U.S. Pat. No. 5,888,365. After solidification, the gel is washed with water to remove residual solvent, and then soaked in a gel buffer solution. The gels were electrophoresed horizontally following conditions described in Shih et al. supra.

(b) Post Electrophoresis Gel Processing:

Gels are briefly rinsed in deionized water, and then fixed for 10 minutes in 10% acetic acid/20% methanol (“fixing solution”). Although effective duration for fixation ranges from a few minutes to days, optimal immunological detection is observed using fixation for 10 to 30 minutes with gel samples containing 200 kDa to 6.5 kDa sized protein.

After fixation, gels are washed in deionized water for 5 to 10 minutes. For detection in situ using antibodies, non-specific binding of the antibody to the gel can be reduced by incubating the gel in 3% non-fat dry milk/0.1×TBN (300 mM Tris base; and 129 mM Boric Acid) for 1 hr at RT. After blocking, gels are briefly rinsed 2-3 times with 1×TS (20 mM Tris HCl; and 150 mM NaCl) and then primary antibody diluted in 1.5% non-fat dry milk/0.1×TBN is added for 1 hr at RT (antibody dilution determined in preliminary experiments). After the primary antibody incubation step, gels are washed 2-3 times with 1×TS for 2-3 min each and a secondary antibody diluted in 1.5% non-fat dry milk/0.1×TBN as recommended by the manufacturer (Goat anti-Rabbit peroxidase conjugated (KPL), 1:1500 to 1:5000) is incubated with the gel for 1 hr at RT. Following secondary antibody, the gel is washed 3-4 times with 1×TST (20 mM Tris HCl; 150 mM NaCl; and 0.05% Tween 20) for 5 min each and the presence of peroxidase detected using TMB membrane peroxidase substrate system (KPL, Cat#: 50-77-00). Color is developed for 1 to 5 min or till a desired result. The gel results can be preserved by scanning the gel in a conventional scanner either at the wet or the dry stages.

(c) Reagents:

The following reagents were used in the examples that follow:

1. Rabbit anti-trypsin inhibitor (Rockland, Cat. #200-4179).

2. Rabbit anti-carbonic anhydrase (Biodesign International, Cat. #W59157R)

3. Rabbit anti-BSA (Sigma, Cat. #: B7276).

4. Rabbit anti-Beta-Galactosidase (E. coli) (Biodesign International, B59136R).

5. Goat anti-Rabbit Ig G peroxidase conjugated (Cat. #: 474-1506, KPL).

6. TMB membrane peroxidase substrate system (KPL, Cat. #: 50-77-00).

7. EZBlue Gel Staining Reagent (Sigma, Cat. #: G1041)

8. E. coli lysate (Promega, Cat. #: S376-A)

d) Proteins:

The following proteins were electrophoresed in gels:

1. Molecular weight markers (high range): Sigma Cat. No. M3788 (36 K-205 K mol. Wt.)

2. Molecular weight markers (wide range): Sigma Cat. No. M4038 (6.5 K-205 K mol. Wt.)

3. Molecular weight markers (broad): Bio-Rad, Cat. #161-0317

### Example 2

Evaluation of Reagent Incubation Conditions for In situ Detection

For a large molecular weight reagent to bind in situ to biomolecules in gels, the reagent needs to diffuse into the gel during the incubation steps. The inventors have identified electrolytes or solvents that when included during incubation with reagent, enhances detectability, possibly through diffusion or other means. Dimethyl sulfoxide (DMSO) and boric acid as enhancers are evaluated below.

An AHT hydrogel (see Example 1) was loaded with 100 and 50 nanograms of SDS-PAGE molecular weight standard (wide range) and electrophoresed as described in Example 1. Following electrophoresis, the gel was cut into 6 strips, each containing two lanes (one containing 100 and the other 50 nanograms of protein), and the gel strips were fixed by incubation at RT for 10 minutes in 10% acetic acid/20% methanol. Incubation of the gel in blocking solution was performed as described in example 1. The steps of incubation with a first reagent (rabbit anti-β-galactosidase at 1:2000) followed by a second reagent (goat anti-rabbit IgG-peroxidase conjugate at 1:3000), both steps conducted for one hour at RT, was performed with the reagents diluted under five different conditions, each condition tested on an individual gel strip;

Condition #1: 1×TS solution (routinely used for Western Blotting);

Condition #2: TST (1×TS containing 0.05% Tween 20);

Condition #3: 1×TS containing 2% DMSO;

Condition #4: 1×TS containing 5% DMSO; and

Condition #5: 0.1×TBN.

The color development was performed as recommended by the manufacture's protocol (Kirkegaard Perry Laboratories, Gaithersburg, Md.) (“KPL”).

Signal/noise ratio analysis showed that 0.1×TBN was superior, providing approximately 10 times greater s/n than 1×TS. The other conditions resulted in s/n that was about 3-5 times lower than 1×TS.

Further experiments were performed as described above except that TBN solutions (0.05×, 0.1×, 0.2×, 0.5× respectively) and 1×TS were used as the first and second antibody diluent. The result indicated that the working solution for TBN ranges from 0.05× to 0.2×, with 0.1× appearing slightly more effective.

Additional experiments were performed as described above except that 0.1×TBN was evaluated and the antibody diluent with various amounts of DMSO or Tween 20 detergent. The results showed that DMSO between 2% to 5% decreased signal/noise by approximately 5 to 10 fold, whereas addition of Tween-20 (0.05%) decreased signal/noise only slightly.

### Example 4

Determination of Blocking Conditions for In situ Gel Detection

Conventional blocking solutions were evaluated for improving signal to noise in AHT hydrogels using the procedure as described in example 1. The results showed that the non-fat dry milk at 3%-5% in 1×TS was an effective blocking reagent, and is somewhat better than 1% bovine serum albumin in 1×TS.

Although blocking can be helpful for in situ gel detection, it is not essential and can be eliminated or compensated for by using a higher concentration of the primary reagent. The background is improved somewhat when a blocking step is used but blocking is not as important as for gel media other than AHT hydrogels because proteins have a very low affinity for this matrix.

### Example 5

In situ-gel Detection of a Large Molecular Weight Protein.

The ability to use in situ detection for large proteins was evaluated for β-galactosidase (116 kDa) which is present in broad molecular weight markers from Bio-Rad (Cat. #: 161-0317). The marker was loaded into wells AHT hydrogel as 1:2 series dilutions containing from 500 to 7.5 nanograms of β-galactosidase. One lane also contained 15 microgram of E. coli lysate (Promega, Cat. #: S376-A). After electrophoresis, the gel was fixed in 10% acetic acid/20% methanol solution for 20 min. In situ detection was performed essentially as a conventional Western blot (see Sambrook et al., supra, 1989) except that both antibodies were diluted into 3% non-fat dry milk/30 mM Tris base, 12.9 mM boric acid. The rabbit anti-beta-galactosidase (Biodesign International, Cat. #: B59136R) at 1:2000 was used as primary antibody. The secondary antibody (KPL, Cat. #: 474-1506), goat anti-rabbit IgG, conjugated with peroxidase was diluted at 1:3000. Color development was performed as recommended by the manufacture's protocol (KPL, Cat. #: 50-77-00). The results showed in situ gel detection of β-galactosidase with a sensitivity down to 7.5 nanograms.

### Example 6

In situ-gel Detection of Small Molecular Weight Proteins.

The ability to use in situ detection for small proteins was evaluated for soybean trypsin inhibitor (21,500 Dalton) and carbonic anhydrase (31,000 Dalton) contained in molecular weight standards. The experimental details were as described in example 5 except that rabbit anti-trypsin inhibitor 1:2000 and rabbit anti-carbonic anhydrase 1:2000 were used as primary antibody. The results showed in situ gel detection of trypsin inhibitor to a sensitivity between 7.5 and 15 nanograms and detection of carbonic anhydrase to a sensitivity of 7.5 nanograms. The sharpness of bands seen in in situ gel detection were similar to that seen when the gel was stained with Coomassie-blue, indicating that limited diffusion of the small proteins in the gel occurred during the protocol used for in-situ gel detection.

### Example 7

Sensitivity of In situ Gel Detection and Western Blotting.

Detection of β-galactosidase was evaluated side-by-side with in situ gel detection and Western blotting. The broad molecular marker (BioRad 161-0317) was loaded as 1:2 series dilutions ranging from 100 to 1.56 nanograms in AHT hydrogels prepared and electrophoresed as described in Example 1.

For Western Blotting, the proteins in the gel were transferred to PVDF membranes using a Bio-Rad mini transfer apparatus as recommended by the manufacturer. The addition of reagent to the gel was performed as described in example 1 except that both primary and secondary antibodies were diluted into 3% non-fat dry milk/20 mM Tris HCl, 150 mM sodium chloride. Rabbit anti-Beta-galactosidase at 1:3000 was used as primary antibody, and Goat anti-Rabbit IgG conjugated with peroxidase at 1:5000 was used as the secondary antibody.

For in situ gel detection, the gel was fixed in 10% acetic acid/20% methanol solution for 20 minutes prior to immunological detection (i.e., antibody incubations). The immunological detection was essentially the same for the Western blot except for the antibody dilution buffer which was 3% non-fat dry milk/30 mM Tris base, 12.9 mM boric acid. Also, a slightly higher concentration of both primary (1:2000) and secondary (1:3000) antibodies were used for in situ detection as compared to the Western blot. The results showed that the sensitivity of the two methods were comparable with detection of as little as 6.5 nanograms of β-galactosidase.

### Example 8

Effect of Incubation Temperature on In Situ Detection with Antibodies.

The electrophoresis conditions and fixation conditions were the same as in example 3. Antibody incubations (rabbit anti-beta galactosidase at 1:2000 and secondary antibodies) were performed for one hour at RT or 37° C. diluted in either 1×TS and 0.1×TBN. Color development was performed using the same temperature and duration as with the antibodies. The results showed a signal/noise ratio of about three to five times greater at 37° C. than at RT for either 1×TS and 0.1×TBN solutions.

### Example 9

In Situ Gel Detection using Biotin/Strepavidin

In situ gel detection was evaluated using the Biotin/Streptavidin system. Biotinylated protein molecular weight markers [Sigma Cat. No. SDS-6B] at 2.5, 5.0, and 10 microliter samples of a 0.1 microgram per microliter stock were loaded on an AHT Hydrogel equilibrated in Tris-borate/ACN buffer and separated by electrophoresis in Tris-glycine buffer. The manufacturer recommends 5 microliter loading for a 10×10 cm minigel, and control experiments showed that a 2.5 microliter sample was near the lowest level of detection by conventional blotting. Following electrophoresis, the gel was removed from the Mylar backing and transferred directly to 20 ml of probe solution containing 0.04 micrograms of peroxidase-labeled streptavidin [Kirkegaard & Perry Laboratories Cat. No. 14-30-00] in 0.5 M sodium thiocyanate [Aldrich Cat. No. 25, 141-0] in water and incubated at room temperature with agitation overnight. The gel was then washed in phosphate-buffered saline solution for two hours with one change of solution, then incubated in TMB peroxidase substrate [Kirkegaard & Perry Laboratories Cat. No. 50-7600] to visualize the bound probe. Molecular weight ladders were rapidly visible in each lane. The level of in situ detection with biotin/streptavidin was comparable to that reported for conventional blotting.

The invention thus has been disclosed broadly and illustrated in reference to representative embodiments described above. Those skilled in the art will recognize that various modifications can be made to the present invention without departing from the spirit and scope thereof. All publications, patent applications, and issued patents, are herein incorporated by reference to the same extent as if each individual publication, patent application or issued patent were specifically and individually indicated to be incorporated by reference in its entirety.

That which is claimed is:

1. A method of detecting biomolecules in situ in gel separation medium, said method comprising:
a) electrophoresing a sample of biomolecules in gel separation media, said media comprising a gellable polymeric material other than cross-linked polyacrylamide;
b) contacting the gel separation media following step a) with
i) a solution comprising at least one detectably labeled reagent directed to a biomolecule under conditions suitable for binding of the reagent to the biomolecule; or
ii) a solution comprising at least one non-detectably labeled reagent directed to a biomolecule under conditions suitable for binding of the reagent to the biomolecule and, a solution comprising at least one detectably labeled reagent directed to the non-detectably labeled reagent under conditions suitable for binding of the detectably labeled reagent to the non-detectably labeled reagent; and
c) detecting the binding of the detectably labeled reagent, indicating detection of biomolecules in the gel.

2. The method of claim 1, wherein said gellable material comprises hydrophilic and hydrophobic domains.

3. The method of claim 1, wherein said gellable material comprises a hydrophilic and hydrophobic multi-block copolymer of partially hydrolyzed polyacrylonitrile.

4. The method of claim 1, wherein said separation media comprises gellable material in a network structure formed through hydrophobic interactions and physical chain entanglement.

5. The method of claim 1, wherein said gellable material is non-covalently cross linked.

6. The method of claim 1, wherein said detectably labeled reagent or said non-detectably labeled reagent is an antibody molecule.

7. The method of claim 1, wherein said detectably labeled reagent or said non-detectably labeled reagent is a polynucleotide or oligonucleotide.

8. The method of claim 1, wherein said detectably labeled reagent or non-detectably labeled reagent has a molecular weight of about 5,000 Daltons or greater.

9. The method of claim 1, wherein said detectably labeled reagent includes a biotin labeled protein or nucleic acid and streptavidin or avidin.

10. The method of claim 1, wherein said detectably labeled reagent is labeled with a detectable moiety selected from the group consisting essentially of an enzyme, fluorochrome, and radioisotope.

11. The method of claim 1, wherein the step of detecting includes the step of contacting the gel with a solution comprising a substrate that becomes colored or changes color following processing by the enzyme.

12. The method of claim 1, wherein in step b), said gel is contacted first with said solution comprising a non-detectably labeled reagent and this is followed by contacting with said solution comprising a detectably labeled reagent.

13. The method of claim 1, wherein in step b)ii), said solution comprising a detectably labeled reagent and said solution comprising a reagent not detectably labeled are combined into a single solution.

14. The method of claim 1, wherein at least one washing step is included.

15. The method of claim 1, wherein said biomolecule detected is present in the gel at about 100 nanograms or less.

16. The method of claim 1, wherein said biomolecule detected in present in the gel at about 10 nanograms or less.

17. The method of claim 1, wherein said sample contains a mixture of different biomolecules.

18. The method of claim 1, wherein said biomolecules are selected from the group consisting essentially of protein, nucleic acid, lipid, or carbohydrate.

19. The method of claim 1, wherein said biomolecules are selected from the group consisting essentially of protein, glycoprotein, lipoprotein or proteoglycan.

20. The method of claim 1, further comprising the step before step b) of fixing the gel following electrophoresis by contacting with an aqueous solution comprising a solvent, an acid or both a solvent and an acid.

21. The method of claim 1, wherein in said step b) said solution further includes dimethylsulfoxide to enhance in situ detection.

22. The method of claim 1, wherein in said step b) said solution further includes boric acid to enhance in situ detection.

23. The method of claim 1, wherein prior to step b), the gel is treated with a solution comprising a substance that reduces background binding of the reagent(s) in the gel.

24. The method of claim 23, wherein said substance that reduces background binding is non-fat dry milk.
